(19) United States
(12) Patent Application Publication
(10) Pub. No.: US 2026/0108763 A1
(43) Pub. Date: Apr. 23, 2026

(54) SUPERFICIAL ELECTRON CONE WITH INTEGRAL ELECTRON BEAM SPOILER PLUG

(71) Applicant: CAMPgear, LLC, Colorado Springs, CO (US)

(72) Inventor: Kiernan McCullough, Colorado Springs, CO (US)

(21) Appl. No.: 19/356,344

(22) Filed: Oct. 13, 2025

Related U.S. Application Data
(60) Provisional application No. 63/710,334, filed on Oct. 22, 2024.

(51) Int. Cl. A61N 5/10

(52) U.S. Cl. CPC A61N 5/1078; A61N 2005/1089

(57) ABSTRACT

A superficial electron cone has a body including a cavity wherein the cavity is closed by a plug at a first end and open at a second end. A superficial cone kit includes a plurality of superficial electron cones of various sizes. The different superficial electron cones are adapted for treating superficial lesions of various sizes.

### RELATED APPLICATION

This application claims the benefit of US Provisional Patent Application Serial No. 63/710,334, filed on October 22, 2024, the full disclosure of which is incorporated herein by reference.

### TECHNICAL FIELD

This document relates generally to a new and improved superficial electron cone adapted to deliver radiation to a superficial lesion of a patient with optimal dose fall off both distally and laterally.

### BACKGROUND

Conventional electron treatments for superficial lesions typically use bolus material to align the electron depth dose curve such that the maximum dose falls at an appropriate depth of treatment and require an additional lateral margin of 1-2 cm to create adequate lateral scattering conditions to fully treat the target. For very small lesions, especially on the face and scalp, creating a bolus device to achieve accurate buildup and appropriate lateral margin is very difficult and requires extensive time and effort crafting specialized devices or relying on advanced 3D printed solutions. In addition, these solutions still require significant lateral margins which ultimately deliver unnecessary additional radiation to the patient for what may be a very small target.

This document relates to a simple and inexpensive superficial electron cone that is adapted to deliver radiation to a superficial lesion of a patient with optimal dose fall off both distally and laterally. Advantageously, this is accomplished while eliminating the need to use bolus material thereby saving the extensive time and effort otherwise required to craft bolus material for each individual application.

### SUMMARY

Each of the following terms written in singular grammatical form: “a”, “an”, and “the”, as used herein, means “at least one”, or “one or more”. Use of the phrase “One or more” herein does not alter this intended meaning of “a”, “an”, or “the”. Accordingly, the terms “a”, “an”, and “the”, as used herein, may also refer to, and encompass, a plurality of the stated entity or object, unless otherwise specifically defined or stated herein, or, unless the context clearly dictates otherwise. For example, the phrase: “a plug”, as used herein, may also refer to, and encompass, a plurality of plugs.

Each of the following terms: “includes”, “including”, “has”, “having”, “comprises”, and “comprising”, and, their linguistic / grammatical variants, derivatives, or/and conjugates, as used herein, means “including, but not limited to”, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof.

The phrase “consisting of”, as used herein, is closed-ended and excludes any element, step, or ingredient not specifically mentioned. The phrase “consisting essentially of”, as used herein, is a semi-closed term indicating that an item is limited to the components specified and those that do not materially affect the basic and novel characteristic(s) of what is specified.

Terms of approximation, such as the terms about, substantially, approximately, etc., as used herein, refers to ± 10 % of the stated numerical value.

In accordance with the purposes and benefits set forth herein, a superficial electron cone, comprises, consists of or consists essentially of a body including a cavity wherein the cavity is closed at a first end and open at a second end. In at least some of the many possible embodiments, the first end of the cavity is closed by a plug having a thickness of about 8 mm to about 12 mm. In some embodiments, the first end of the cavity is closed by a plug having a thickness of about 10 mm.

In at least some embodiments, the body is a cylindrical wall having an interior diameter of about 15 mm and a length of about 110 mm. In at least some embodiments, the body and the plug are made from acrylic and the cylindrical wall has a thickness of between about 2.5 and about 4.0 mm. In some embodiments, the body is a cylindrical wall having an interior diameter of about 20 mm and a length of about 110 mm. In some embodiments, the body is a cylindrical wall having an interior diameter of about 25 mm and a length of about 110 mm. In some embodiments, the body is a cylindrical wall having an interior diameter of about 30 mm and a length of about 120 mm. In other embodiments, the body is a cylindrical wall having an interior diameter of about 35 mm and a length of about 125 mm.

In accordance with yet another aspect, a superficial electron cone kit, comprises, consists of or consists essentially of:

(1) a first superficial electron cone having a first body including a first cylindrical sidewall (a) having an interior diameter of about 15 mm and a length of about 110 mm and (b) defining a first cavity wherein the first cavity is closed at a first end by a plug having a thickness of about 8 mm to about 12 mm and open at a second end;

(2) a second superficial electron cone having a second body including a second cylindrical sidewall (a) having an interior diameter of about 20 mm and a length of about 110 mm and (b) defining a second cavity wherein the second cavity is closed at a first end by a plug having a thickness of about 8 mm to about 12 mm and open at a second end; and

(3) a third superficial electron cone having a third body including a third cylindrical sidewall (a) having an interior diameter of about 25 mm and a length of about 110 mm and (b) defining a third cavity wherein the third cavity is closed at a first end by a plug having a thickness of about 8 mm to about 12 mm and open at a second end.

The superficial electron cone kit may further include a fourth superficial electron cone having a fourth body including a fourth cylindrical sidewall (a) having an interior diameter of about 30 mm and a length of about 120 mm and (b) defining a fourth cavity wherein the fourth cavity is closed at a first end by a plug having a thickness of about 8 mm to about 12 mm and open at a second end.

The superficial electron cone kit may further include a fifth superficial electron cone having a fifth body including a fifth cylindrical sidewall (a) having an interior diameter of about 35 mm and a length of about 125 mm and (b) defining a fifth cavity wherein the fifth cavity is closed at a first end by a plug having a thickness of about 8 mm to about 12 mm and open at a second end.

In accordance with yet another aspect, a method of securing a superficial electron cone to an electron tray of a linear accelerator, comprises, consists of or consists essentially of:

(1) positioning a body of the superficial electron cone onto the electron tray with a plug end of the body adjacent the electron tray;

(2) pouring blocking material into the electron tray around the body; and

(3) allowing the blocking material to set in the electron tray around the body.

The method may further include applying a strip of masking tape around the body before the positioning of the body onto the electron tray. The method may further include applying a weight onto the top of the body before the pouring of the blocking material in order to better secure the body in position during the pouring.

In at least some embodiments, the method includes selecting the superficial electron cone from a plurality of electron cones of different size before positioning the body of the superficial electron cone onto the electron tray.

In the following description, there are shown and described several different embodiments of the new and improved superficial electron cone, the superficial electron cone kit and the related method of securing a superficial electron cone to an electron tray of a linear accelerator. As it should be realized, the apparatus and method are capable of other, different embodiments and their several details are capable of modification in various, obvious aspects all without departing from the apparatus and method as set forth and described in the following claims. Accordingly, the drawings and descriptions should be regarded as illustrative in nature and not as restrictive.

### BRIEF DESCRIPTION OF THE DRAWING FIGURES

The accompanying drawing figures incorporated herein by reference and forming a part of the specification, illustrate several aspects of the new and improved apparatus and method and together with the description serve to explain certain principles thereof.

FIG. 1 is a perspective view of a superficial electron cone.

FIG. 2 is a perspective view of a superficial electron cone kit comprising five electron cones of the type shown in FIG. 1 but of different sizes.

FIG. 3 is a graph of a 2.5 cm cone ionization curve.

FIG. 4 is a graph depicting the radial dose profile of a 2.5cm cone.

FIGS. 5A-5C are a series of perspective views illustrating a method of securing a superficial electron cone to an electron tray of a linear accelerator. More specifically, FIG. 5A illustrates taping a plug end of the cone. FIG. 5B illustrates positioning the taped cone on the electron tray. FIG. 5C illustrates pouring blocking material into the electron tray around the body of the cone.

FIG. 6 illustrates the cone properly secured to the electron tray and ready for use.

FIG. 7 illustrates the mounting of the electron tray and superficial cone onto a linear accelerator.

Reference will now be made in detail to the presently preferred embodiments of the superficial electron cone, the superficial electron cone kit and the related method of securing a superficial electron cone to an electron tray of a linear accelerator.

### DETAILED DESCRIPTION

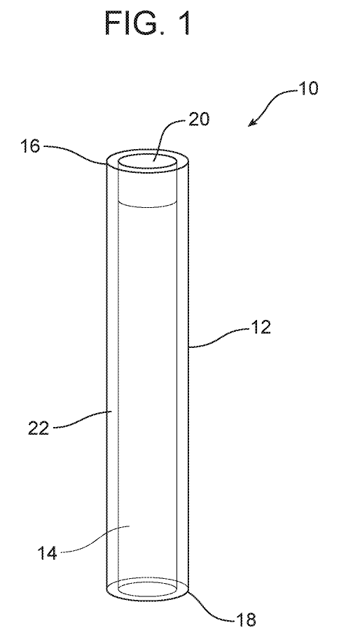

Reference is now made to FIG. 1 illustrating the new and improved superficial electron cone 10. The superficial electron cone 10 comprises a body 12 that includes a cavity or blind bore 14 that is closed at a first end 16 and open at a second, opposite end 18. In the illustrated embodiment, the body 12 is in the shape of an elongated cylinder. It should be appreciated that the body 12 may comprise other shapes that are adapted to direct an electron beam toward a targeted superficial lesion of a patient.

In the illustrated embodiment, the first end of the body is closed by a plug 20. The plug 20 may be integrally formed with the body 12 or secured in place within the first end of the body by any appropriate means (e.g. adhesive, heat welding). Both the body 12 and the plug 20 are made from an electron beam spoiling material. In at least some embodiments, both the body 12 and the plug 20 are made from acrylic. In one possible embodiment, the cylindrical wall 22 of the body 12 has a thickness of between 2.5 and 4.0 mm and the plug 20 has a thickness of between about 8 mm and about 12 mm. In one particularly useful embodiment, the plug 20 has a thickness of about 10 mm.

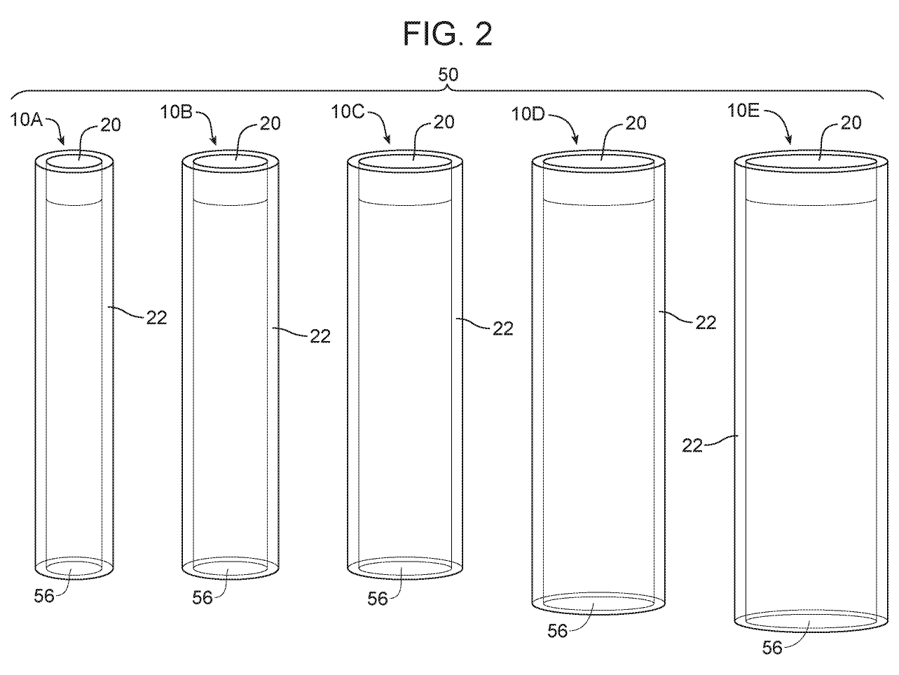

FIG. 2 illustrates a superficial cone kit 50, comprising a series of five superficial cones 10A, 10B, 10C, 10D and 10E, all of different sizes. Each cone 10A-10E includes a cylindrical wall 22 and a plug 20 at the first end that fully closes the internal cavity 56. In one possible embodiment, the first cone 10A has a length of about 110 mm, an interior diameter of about 15 mm, a plug thickness of about 10 mm and a cylindrical wall diameter of about 3 mm. The second cone 10B has a length of about 110 mm, an interior diameter of about 20 mm, a plug thickness of about 10 mm and a cylindrical wall diameter of about 3 mm. The third cone 10C has a length of about 110 mm, an interior diameter of about 25 mm, a plug thickness of about 10 mm and a cylindrical wall diameter of about 3 mm. The fourth cone 10D has a length of about 120 mm, an interior diameter of about 30 mm, a plug thickness of about 10 mm and a cylindrical wall diameter of about 3 mm. Finally, the fifth cone 10E has a length of about 125 mm, an interior diameter of about 35 mm, a plug thickness of about 10 mm and a cylindrical wall diameter of about 3 mm. The different size cones 10A-10E accommodate superficial legion targets of various sizes.

Each superficial electron cone 10A, 10B, 10C, 10D and 10E is specially designed to deliver radiation to the patient’s surface with optimal dose fall off both distally and laterally.

Advantageously, the superficial electron cone 10, 10A-10E is able to operate without the use of bolus by scattering the electrons upstream of the patient using the acrylic plug 20. The resulting depth dose curve is near maximum at the patient surface and a near uniform dose is deposited until a depth of ~5mm before decreasing quickly. This has the additional benefit of flattening the radiation profile such that everything within the cone receives a therapeutic dose, reducing the need for additional lateral margins. With these two characteristics, the superficial cone 10, 10A-10E is very easy to use for otherwise difficult clinical setups.

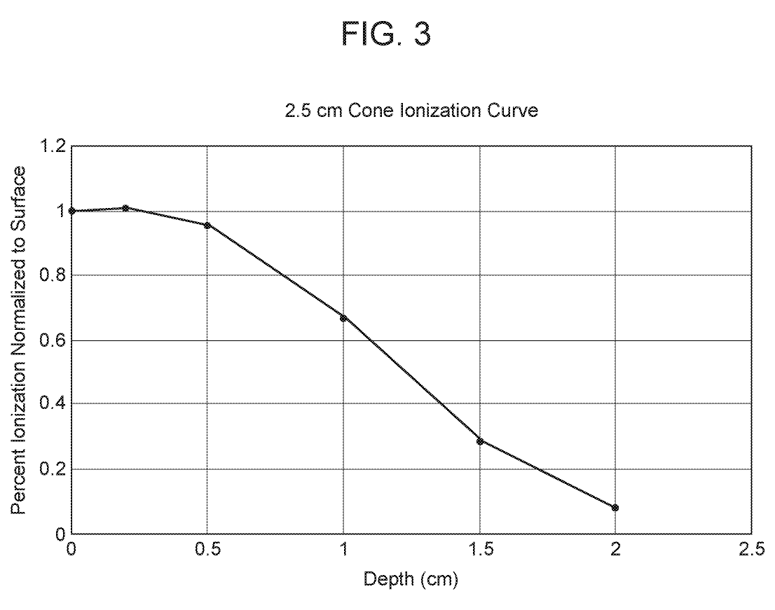

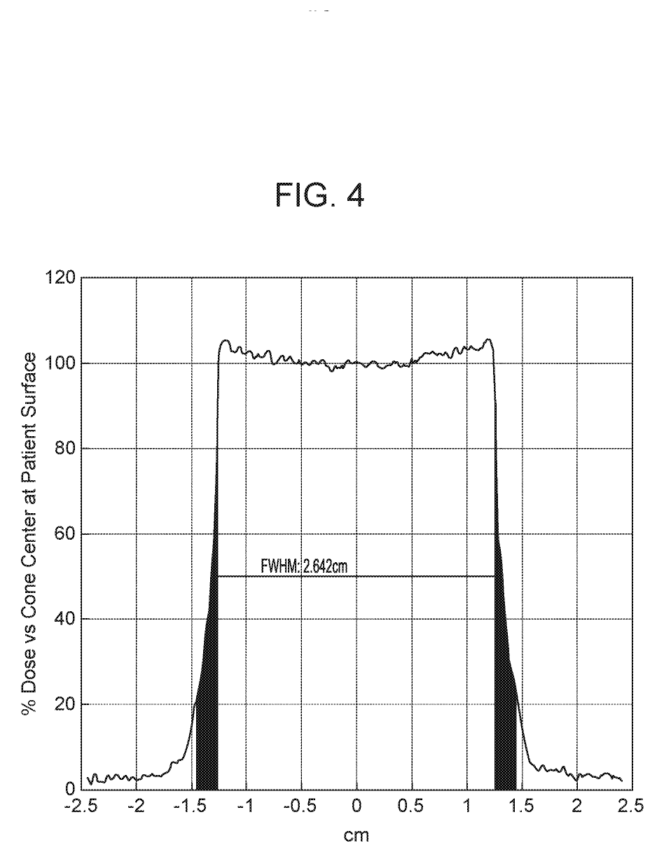

As noted above, the superficial cone 10, 10A-10E is essentially an electron “beam-spoiling” device that moderates a 6 MeV electron beam, which is conventionally available on most linear accelerators used for cancer therapy. The location of the acrylic plug 20 in conjunction with the length of the cylindrical sidewall 22 are specifically adapted to generate an optimal beam energy and uniform distribution for ideal treatment conditions. Characteristics of the radiation profile are shown for the 25 mm cone in FIG. 3 (ionization curve) and FIG. 4 (radial dose profile). Cone dimensions 10A-10E have been designed to closely replicate the same treatment conditions across all cone diameters.

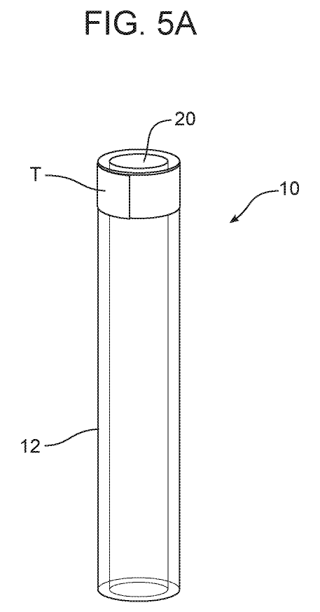

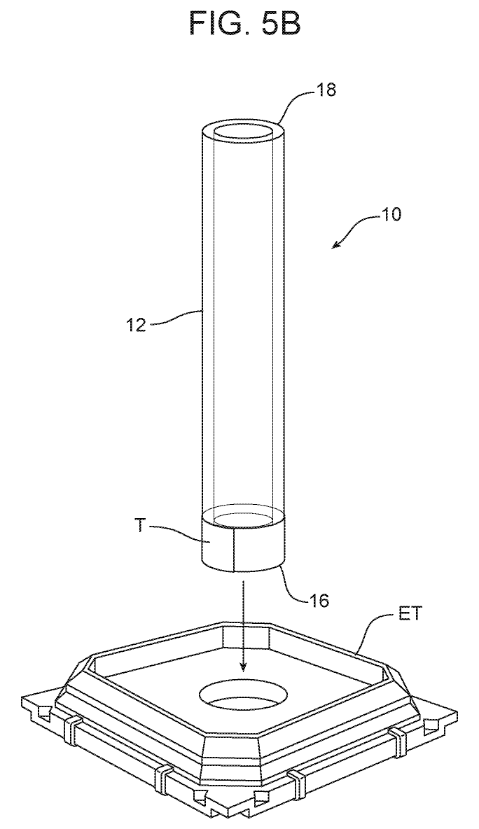

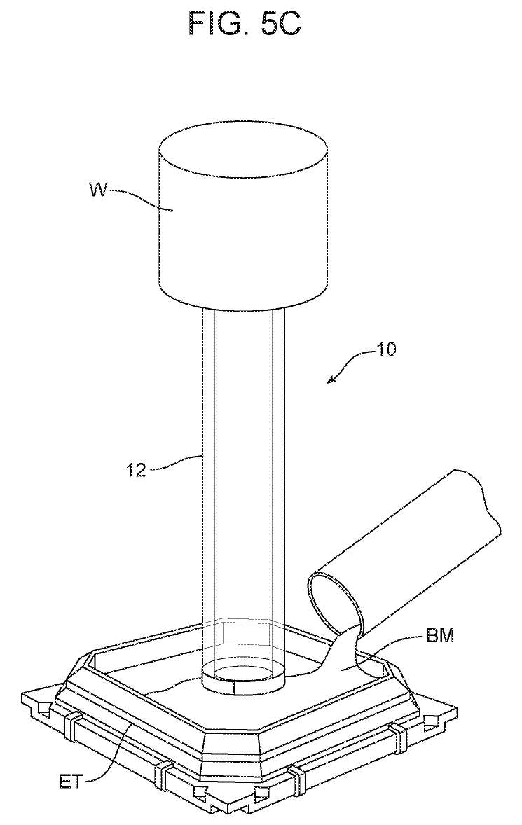

Advantageously, the superficial cones 10, 10A-10E may be easily integrated for use with substantially any available linear accelerator LA adapted for delivering an electron beam for the treatment of a superficial lesion of a patient. Reference is now made to FIGS. 5A-5C which illustrate a method of securing a superficial electron cone 10 to an electron tray ET of a linear accelerator LA. The method includes selecting a superficial electron cone 10, 10A-10E of desired size for treating a superficial lesion of the patient. One then applies a strip of masking tape T around the first end of the body 12 that includes the plug 20 (see FIG. 5A) before the positioning of the body onto the center of the electron tray ET (see FIG. 5B). Note how the end of the cone 10 with the solid acrylic plug 20 is oriented with respect to the electron tray ET. In this way, in use, the plug end 16 of the cone 10 will be oriented toward the beam source while the open end 18 of the cone 10 will be oriented toward the patient.

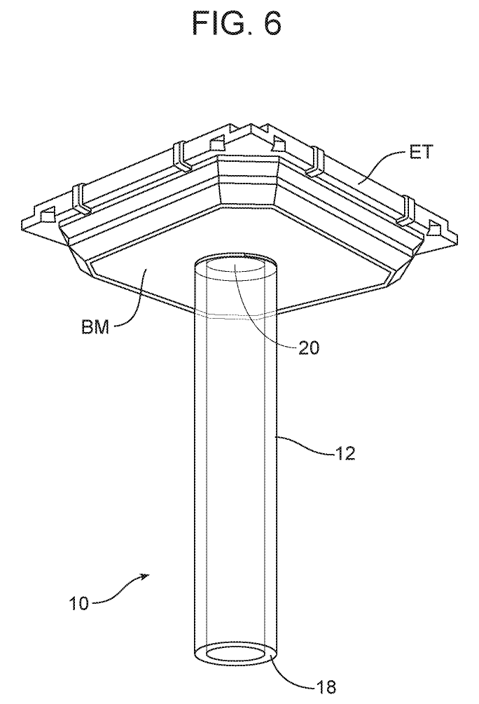

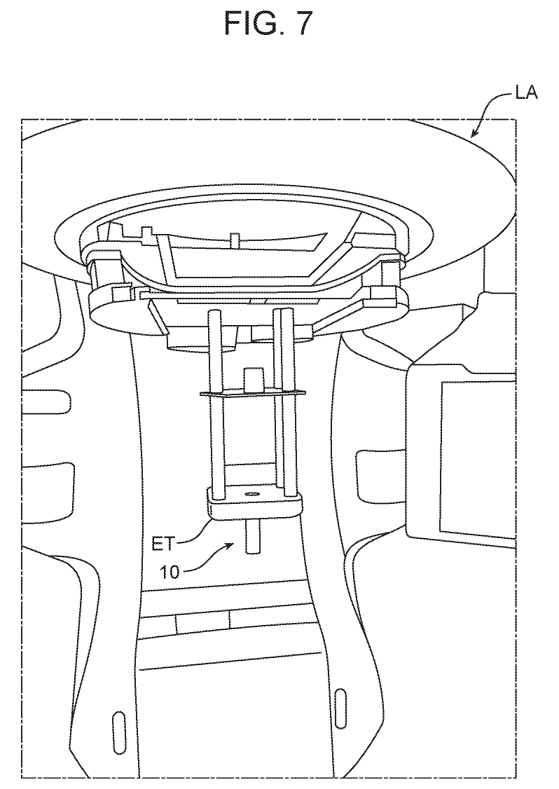

Next one pores a blocking material BM, such as Cerrobend, into the electron tray ET around the body 12 as shown in FIG. 5C. As shown a weight W may be applied onto the top of the body 12 before the pouring of the blocking material BM in order to better secure the body in position during the pouring. Once the blocking material BM sets in the electron tray ET around the body 12, the cone 10 will not spin, loosen or move. FIG. 6 illustrates the cone 10 properly secured by the blocking material BM in the electron tray ET. FIG. 7 illustrates the electron tray ET and cone 10 attached to the linear accelerator LA in a manner known in the art.

Although the superficial electron cone 10, 10A-10E, the superficial electron cone kit 50 and the related method of this disclosure have been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

What is claimed:

1. A superficial electron cone, comprising:
a body including a cavity wherein the cavity is closed at a first end and open at a second end.

2. The superficial electron cone of claim 1, wherein the first end of the cavity is closed by a plug having a thickness of about 8 mm to about 12 mm.

3. The superficial electron cone of claim 1, wherein the first end of the cavity is closed by a plug having a thickness of about 10 mm.

4. The superficial electron cone of claim 3, wherein the body is a cylindrical wall having an interior diameter of about 15 mm and a length of about 110 mm.

5. The superficial electron cone of claim 4, wherein the body and the plug are made from acrylic and the cylindrical wall has a thickness of between about 2.5 and about 4.0 mm.

6. The superficial electron cone of claim 3, wherein the body is a cylindrical wall having an interior diameter of about 20 mm and a length of about 110 mm.

7. The superficial electron cone of claim 6, wherein the body and the plug are made from acrylic and the cylindrical wall has a thickness of between about 2.5 and about 4.0 mm.

8. The superficial electron cone of claim 3, wherein the body is a cylindrical wall having an interior diameter of about 25 mm and a length of about 110 mm.

9. The superficial electron cone of claim 8, wherein the body and the plug are made from acrylic and the cylindrical wall has a thickness of between about 2.5 and about 4.0 mm.

10. The superficial electron cone of claim 3, wherein the body is a cylindrical wall having an interior diameter of about 30 mm and a length of about 120 mm.

11. The superficial electron cone of claim 4, wherein the body and the plug are made from acrylic and the cylindrical wall has a thickness of between about 2.5 and about 4.0 mm.

12. The superficial electron cone of claim 3, wherein the body is a cylindrical wall having an interior diameter of about 35 mm and a length of about 125 mm.

13. The superficial electron cone of claim 4, wherein the body and the plug are made from acrylic and the cylindrical wall has a thickness of between about 2.5 and about 4.0 mm.

14. The superficial electron cone of claim 2, wherein the body and the plug are made from acrylic.

15. A superficial electron cone kit, comprising:
a first superficial electron cone having a first body including a first cylindrical sidewall (a) having an interior diameter of about 15 mm and a length of about 110 mm and (b) defining a first cavity wherein the first cavity is closed at a first end by a plug having a thickness of about 8 mm to about 12 mm and open at a second end;
a second superficial electron cone having a second body including a second cylindrical sidewall (a) having an interior diameter of about 20 mm and a length of about 110 mm and (b) defining a second cavity wherein the second cavity is closed at a first end by a plug having a thickness of about 8 mm to about 12 mm and open at a second end; and
a third superficial electron cone having a third body including a third cylindrical sidewall (a) having an interior diameter of about 25 mm and a length of about 110 mm and (b) defining a third cavity wherein the third cavity is closed at a first end by a plug having a thickness of about 8 mm to about 12 mm and open at a second end.

16. The superficial electron cone kit of claim 15, further including a fourth superficial electron cone having a fourth body including a fourth cylindrical sidewall (a) having an interior diameter of about 30 mm and a length of about 120 mm and (b) defining a fourth cavity wherein the fourth cavity is closed at a first end by a plug having a thickness of about 8 mm to about 12 mm and open at a second end.

17. The superficial electron cone kit of claim 16, further including a fifth superficial electron cone having a fifth body including a fifth cylindrical sidewall (a) having an interior diameter of about 35 mm and a length of about 125 mm and (b) defining a fifth cavity wherein the fifth cavity is closed at a first end by a plug having a thickness of about 8 mm to about 12 mm and open at a second end.

18. A method of securing a superficial electron cone to an electron tray of a linear accelerator, comprising:
positioning a body of the superficial electron cone onto the electron tray with a plug end of the body adjacent the electron tray;
pouring blocking material into the electron tray around the body; and
allowing the blocking material to set in the electron tray around the body.

19. The method of claim 18, further including applying a strip of masking tape around the body before the positioning of the body onto the electron tray.

20. The method of claim 19, further including applying a weight onto the top of the body before the pouring of the blocking material in order to secure the body in position during the pouring.
